(19) United States
(12) Patent Application Publication
(10) Pub. No.: US 2025/0082882 A1
(43) Pub. Date: Mar. 13, 2025

(54) Devices and Methods for Monitoring Respiration of a Tracheostomy Patient

(71) Applicant: THE REGENTS OF THE UNIVERSITY OF CALIFORNIA, Oakland, CA (US)

(72) Inventors: John J. Clement, Sacramento, CA (US); Nicole Del Rosario Narag, Sacramento, CA (US)

(21) Appl. No.: 18/291,638

(22) PCT Filed: Jul. 20, 2022
(86) PCT No.: PCT/US2022/037738

§ 371 Date: Jan. 24, 2024

Related U.S. Application Data
(60) Provisional application No. 63/226,547, filed on Jul. 28, 2021.

(51) Int. Cl. A61M 16/04; A61M 16/00; A61M 16/08

(52) U.S. Cl. CPC A61M 16/0463; A61M 16/0858; A61M 2016/0027; A61M 2205/3334

(57) ABSTRACT

Provided herein are heat moisture exchanger (HME) systems and sleep study systems for performing sleep studies of patients with tracheostomy tubes. The HME systems provided herein may include a monitoring tube to enable performance of sleep studies of patients with tracheostomies. The HME systems provided herein may include a housing assembly that defines a central cavity which is in fluid communication with a tracheostomy port for coupling to a hub of a tracheostomy tube of a patient. The HME systems provided herein may also include an HME filter that is positioned within the housing assembly between an inhalation/exhalation opening and the central cavity. The monitoring tube extends from outside of the housing assembly into the housing assembly and may be connected to a respiration monitoring device for detecting respiration parameters of the patient during the sleep study. Methods for performing sleep studies are also provided herein.

### CROSS-REFERENCES TO RELATED APPLICATIONS

This application claims priority to U.S. Provisional Patent Application No. 63/226,547, filed Jul. 28, 2021, the contents of which are hereby incorporated by reference in their entirety for all purposes.

### BACKGROUND

Tracheostomy is a surgical procedure that may be performed by creating an opening on the anterior side of a patient's neck, bypassing the upper airway and providing direct access to the trachea. Tracheostomy is performed, for example, to bypass an upper airway obstruction, to mitigate complications of vocal cord pathology, or to more safely provide long-term mechanical ventilation. Common complications of tracheostomy include impaired humidification, warming, and filtering of inhaled air. These impairments correspond to functions that ordinarily occur when air passes through the upper airway as the patient breathes.

During sleep, insufficient humidification, warming, and/or filtering of air may cause discomfort for a tracheostomy patient, and it is beneficial for the patient to use a heat moisture exchanger (HME) coupled to the hub of the tracheostomy tube in order for inhaled air to be substantially the same temperature and humidity as if the inhaled air were inhaled through the upper airway. HMEs may additionally remove particles from inhaled air.

Tracheostomy patients may encounter sleep disorders similar to non-tracheostomy patients. Non-tracheostomy patients encountering sleep disorders may be subjects of a sleep study, for example polysomnography. During a sleep study, a non-tracheostomy patient's respiratory function may be monitored using a nasal cannula inserted into the patient's nostrils. However, since tracheostomy patients have limited or no respiratory airflow through the nostrils, a nasal cannula cannot be used to monitor respiratory function. Accordingly, there is a need to be able to monitor the respiratory function of tracheostomy patients during sleep studies. The present invention satisfies this need, and provides related advantages as well.

### BRIEF SUMMARY

In a first aspect, provided herein are heat moisture exchanger (HME) systems, e.g., for use during a sleep study (e.g., polysomnography) of a patient (e.g., a patient who has a tracheostomy). The HME systems disclosed herein may comprise: (a) a housing assembly defining a central cavity and at least one opening, and comprising a tracheostomy port configured to couple to a hub of a tracheostomy tube of the patient, wherein the tracheostomy port is in direct fluidic communication with the central cavity; (b) at least one HME filter positioned within the housing assembly between the at least one opening and the central cavity, wherein the at least one HME filter is configured to absorb heat and moisture of exhalations from the patient in order to warm and humidify inhalations of the patient flowing from the at least one opening through the HME filter to the tracheostomy port; and (c) a monitoring tube extending from outside of the housing assembly into the housing assembly, wherein the monitoring tube comprises an open end positioned within the central cavity, and wherein the monitoring tube is configured to be connected to a respiration monitoring device in order to detect respiration parameters of the patient during the sleep study.

In some embodiments, the at least one HME filter comprises a first HME filter and a second HME filter, the at least one opening comprises a first opening and a second opening, the first HME filter is positioned adjacent to the first opening and the second HME filter is positioned adjacent to the second opening, and the first HME filter and second HME filter are positioned on opposite sides of the central cavity so that the open end of the monitoring tube is positioned between the first HME filter and the second HME filter.

In some embodiments, the housing assembly further comprises a main body defining the central cavity, and an end cap coupled to the main body adjacent to the first HME filter, wherein the end cap defines a sleeve through which the monitoring tube extends. In particular embodiments, the monitoring tube extends from the sleeve to the central cavity between the first HME filter and an inner sidewall of the main body. In some instances, the first HME filter is cylindrical and defines a channel, and the monitoring tube is positioned within the channel.

In some embodiments, the housing assembly further comprises a main body defining the central cavity and the monitoring tube extends through a sidewall of the main body into the central cavity. In particular embodiments, the main body comprises a sleeve defining a curved bend and the monitoring tube extends through the sleeve.

In some embodiments, the at least one HME filter comprises a first HME filter, the housing assembly further comprises a main body defining the central cavity, the first HME filter is positioned within the main body, the housing assembly further comprises an end cap coupled to the main body adjacent to the first HME filter, and the monitoring tube extends through a sidewall of the main body into the central cavity. In particular embodiments, the main body defines a first cylindrical portion, in which the first HME filter is positioned, and a second cylindrical portion, narrower than the first cylindrical portion, defining the central cavity, and the monitoring tube extends through a sidewall of the second cylindrical portion into the central cavity.

In some embodiments, the tracheostomy port defines an axis and the monitoring tube extends away from the housing assembly in a direction approximately perpendicular (e.g. +/−10 degrees of perpendicular) to the axis of the tracheostomy port. In other embodiments, the main body defines a suction port and a cap removably coupled to the suction port configured to allow for removal of secretions from the central cavity.

In some embodiments, the monitoring tube further comprises at least one connector at an end opposite to the open end within the central cavity and the connector is configured to connect the monitoring tube to the respiration monitoring device. In some instances, the monitoring tube further comprises a bifurcation, the at least one connector comprises a first connector and a second connector, and the first connector is configured to connect to an end tidal carbon dioxide (ETCO2) detector and the second connector is configured to connect to a pressure transducer to measure inhalation and exhalation airflow.

In a second aspect, provided herein are sleep study systems. The sleep study systems provided herein are useful, for example, for performing a sleep study (e.g., polysomnography) of a patient (e.g., a patient who has a tracheostomy). The sleep study systems provided herein may comprise an HME system described herein and at least one respiration monitoring device. In some embodiments, the respiration monitoring device is fluidically coupled to the monitoring tube and configured to monitor respiration of the patient. In some embodiments, the at least one respiration monitoring device comprises an end tidal carbon dioxide (ETCO2) detector and/or a pressure transducer configured to measure inhalation and exhalation airflow of the patient.

In some embodiments, the sleep study system further comprises a processor unit, and the at least one respiration monitoring device is connected to the processor unit. In other embodiments, the sleep study system further comprises at least one of an electroencephalogram (EEG) monitor, an electrooculogram (EOG) monitor, a pulse oximeter, and an electrocardiogram (ECG) monitor connected to the processor unit.

In a third aspect, provided herein are methods for performing a sleep study of a patient (e.g., with a tracheostomy tube). The methods provided herein may comprise: (a) coupling the tracheostomy port of an HME system described herein to a hub of the tracheostomy tube of the patient; (b) coupling the monitoring tube to at least one of an end tidal carbon dioxide detector and a pressure transducer; and (c) detecting the end tidal carbon dioxide and/or inhalation and exhalation airflow of the patient as the patient spontaneously breathes with the end tidal carbon dioxide detector and/or the pressure transducer.

In some embodiments, the monitoring tube is bifurcated and the method further comprises coupling the monitoring tube to both the end tidal carbon dioxide detector and the pressure transducer. In other embodiments, the method further comprises connecting the end tidal carbon dioxide detector and/or the pressure transducer to a processor unit. In some embodiments, the method further comprises connecting an electroencephalogram (EEG) monitor, an electrooculogram (EOG) monitor, a pulse oximeter, and/or an electrocardiogram (ECG) monitor to the patient and a processor unit.

This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings, and each claim.

The foregoing, together with other features and examples, will be described in more detail below in the following specification, claims, and accompanying drawings.

### BRIEF DESCRIPTION OF THE DRAWINGS

The features of the various embodiments described above, as well as other features and advantages of certain embodiments of the present invention will be more apparent from the following detailed description taken in conjunction with the accompanying drawings, in which:

FIGS. 1A-1G show views of an HME system for monitoring respiration of a tracheostomy patient including two HME filters and a monitoring tube extending through an open end of a housing assembly, according to some embodiments of the present invention.

FIGS. 2A and 2B show an HME filter, according to some embodiments of the present invention.

FIG. 3 shows an end cap of a housing assembly of an HME system, according to some embodiments of the present invention.

FIGS. 4A-4F show views of an HME system for monitoring respiration of a tracheostomy patient including two HME filters and a monitoring tube extending through a sidewall of a main body of a housing assembly, according to some embodiments of the present invention.

FIGS. 5A-5E show views of an HME system for monitoring respiration of a tracheostomy patient including an HME filter and a monitoring tube extending through a sidewall of a main body of a housing assembly, according to some embodiments of the present invention.

FIG. 6 shows an assembly of an HME system and bifurcated monitoring tube, according to some embodiments of the present invention.

FIG. 7 shows a sleep study system including an HME system, according to some embodiments of the present invention.

Throughout the drawings, it should be noted that like reference numbers are typically used to depict the same or similar elements, features, and structures.

### DETAILED DESCRIPTION

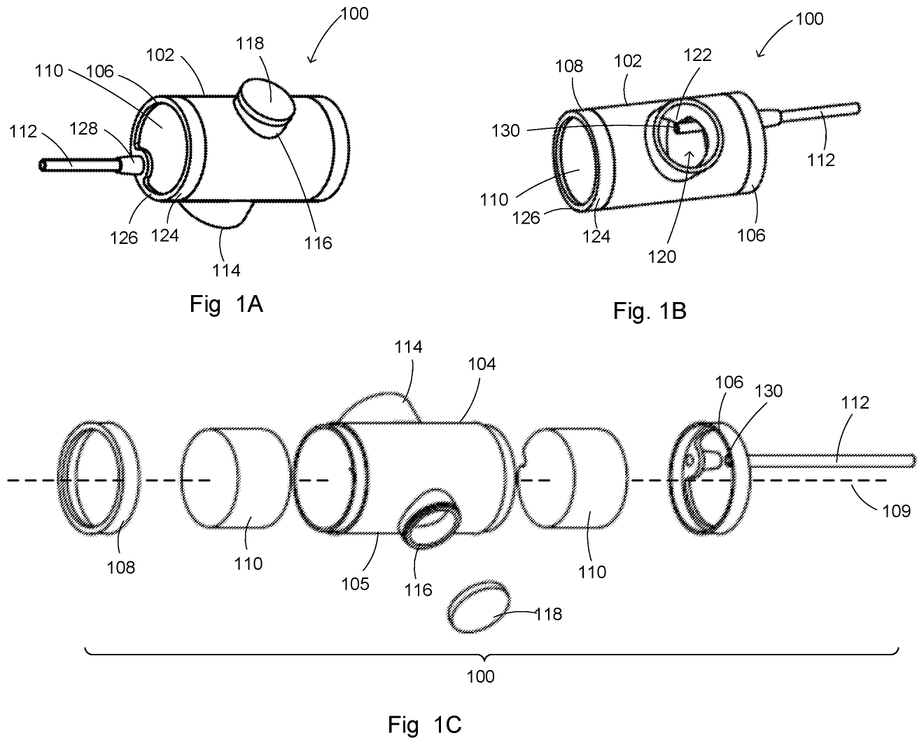

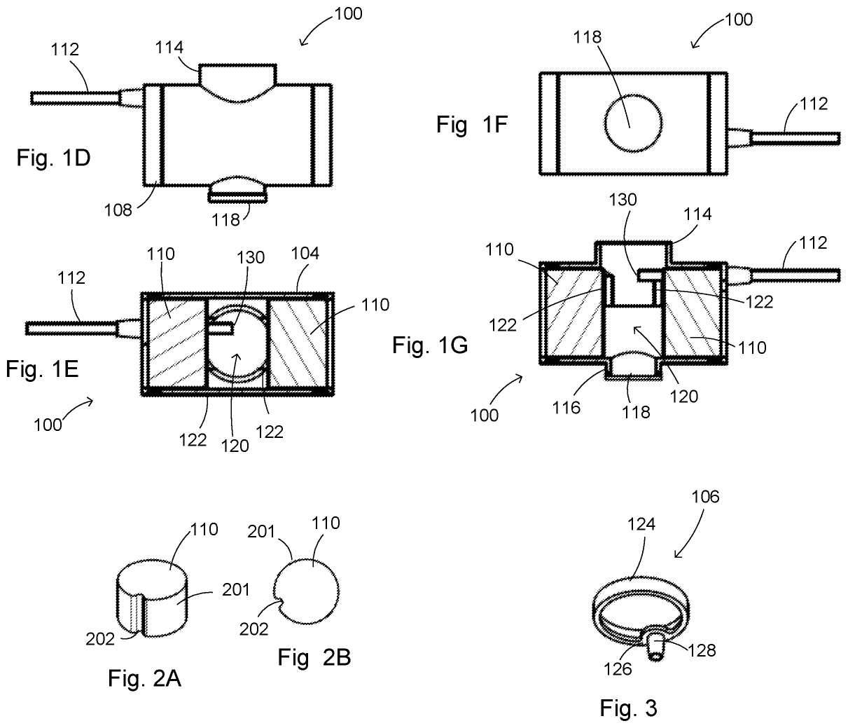

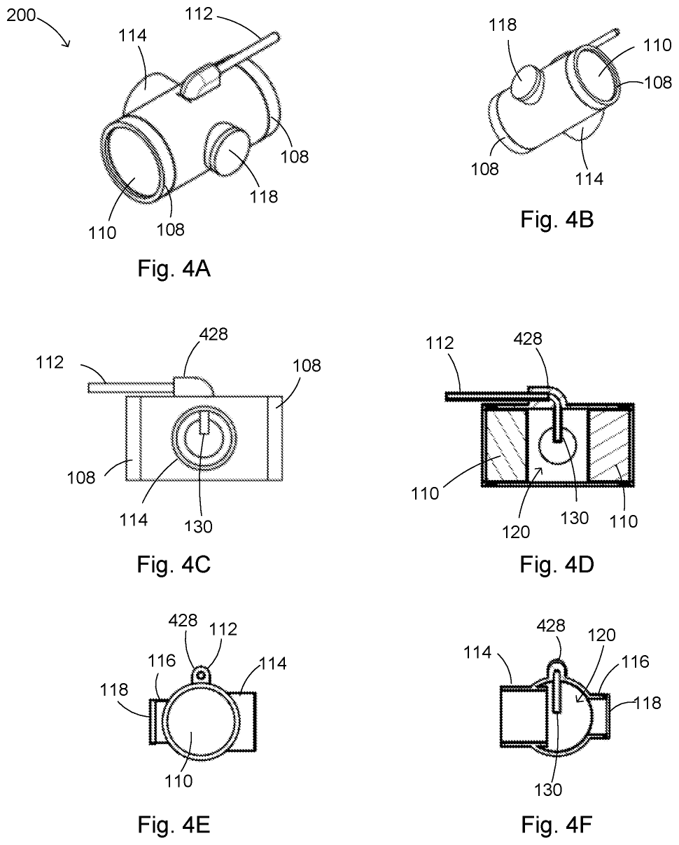

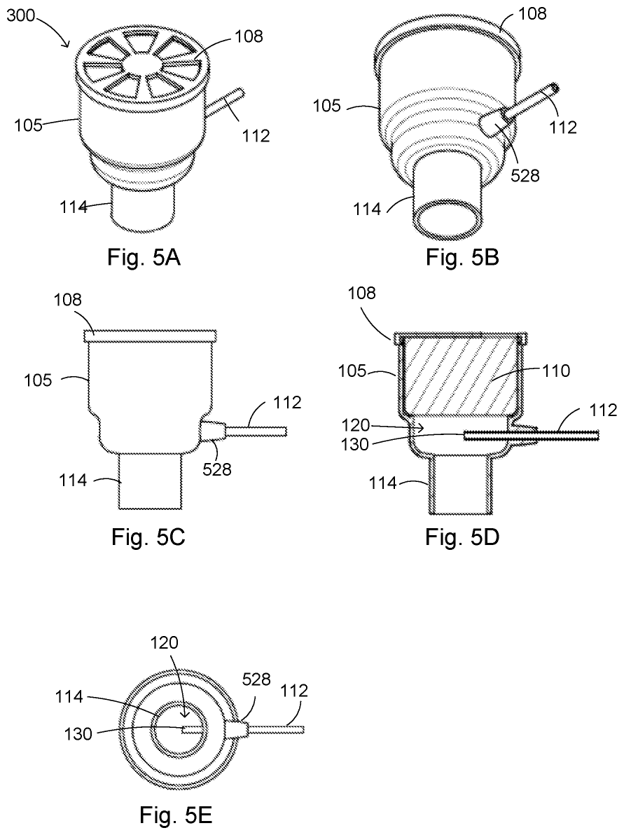

Aspects of the present invention relate generally to HME systems for monitoring respiration of a patient with a tracheostomy, for example as shown in FIGS. 1A-1G, 4A-4F, and 5A-5E. In some embodiments, the HME systems include a housing assembly comprising a main body defining a tracheostomy port for connecting to the hub of a tracheostomy tube. One or more HME filters may be positioned within the housing assembly, for example one HME filter as shown in FIGS. 5A-5E or two HME filters as shown in FIGS. 1A-1G and 4A-4F. The housing assembly may include one or more end caps coupled to the main body, and the main body may include retaining features for maintaining the position of the one or more HME filters within the housing assembly. Inhalations from the patient are drawn through the HME system by first being drawn through one or more openings in the housing assembly, then through the one or more HME filters, then through a central cavity of the housing cavity, then through the tracheostomy port and into the patient's tracheostomy tube. Exhalations from the patient may follow a reverse path, wherein the exhalations pass from the tracheostomy tube, through the tracheostomy port, through the internal cavity, through the one or more HME filters, and out of the HME system through the one or more openings of the housing assembly. The HME system further includes a monitoring tube extending from outside of the housing assembly into the central cavity. The monitoring tube may be fluidically connected to one or more respiration monitoring devices or systems in order to monitor the patient's respiration during a sleep study. Non-limiting examples of suitable respiration monitoring devices include end tidal carbon dioxide monitors and pressure transducers (e.g., used to measure inhalation and exhalation airflow).

The following high-level summary is intended to provide a basic understanding of some of the novel innovations depicted in the figures and presented in the corresponding descriptions provided below.

FIGS. 1A-1G show an embodiment of an HME system 100, for monitoring respiration of a patient with a tracheostomy, as described above. As shown, in some embodiments an HME system 100 may include a housing assembly 102 comprising a main body 104, a first end cap 106, and a second end cap 108. As shown, the HME system 100 may further include two HME filters 110. Additionally, as shown, the HME system 100 may also include a monitoring tube 112 extending from outside of the housing assembly 102 into the housing assembly 102.

In some embodiments, for example as shown in FIG. 1A, the housing assembly 102 may be generally cylindrical around a longitudinal axis 109. In particular embodiments, for example as shown, the main body 104 may include a first cylindrical portion 105 extending parallel to the longitudinal axis 109, and a second cylindrical section, defining a tracheostomy port 114, extending approximately perpendicular to the longitudinal axis 109. In other words, the main body 104 may be approximately T-shaped. The tracheostomy port 114 may be connectable to a hub of a tracheostomy tube in order to fluidically connect the HME system to the airway of a patient. In some embodiments, for example as shown, the tracheostomy port 114 is located centrally on the first cylindrical portion 105.

The main body 104 may further include a third cylindrical portion, defining a suction port 116, extending from the first cylindrical portion 105 approximately perpendicularly to the longitudinal axis 109. In other words, the main body may be approximately plus-sign shaped. The housing assembly 102 may also include a suction cap 118, removably coupled to the suction port 116 in order to prevent respiration flowing through the suction port 116 during normal use. The suction cap 118 may couple with an annular snap-fit or press-fit and may be removed during use in order to access a central cavity 120 of the housing assembly 102 in order to periodically remove patient secretions from the central cavity 120. In some embodiments, the housing assembly 102 may also include an oxygen port for connecting to an oxygen supply to deliver supplemental oxygen to the patient.

The central cavity 120 is defined within the main body 104 and is in fluidic communication with the tracheostomy port 114. In some embodiments, the central cavity 120 is defined in a central portion of the first cylindrical portion 105 and is in direct fluid communication with the tracheostomy port 114 and the suction port 116, as shown for example in the cross-sectional view of FIG. 1G. The HME filters 110 may be positioned between ends of the main body 104 and the central cavity 120, as shown for example in the cross-sectional view of FIG. 1G. Portions of the second cylindrical section, tracheostomy port 114, may extend into the central cavity 120 in order to define tabs 122 with the central cavity 120. The tabs 122 may engage with the inner faces of the HME filters 110 in order to maintain the position of the HME filters 110 within the main body 104.

The housing assembly 102 may include end caps 106 and 108 removably coupled to ends of the main body 104. The end caps 106 and 108 may each define a sidewall 124, and an end flange 126 coupled to the sidewall 124. The sidewalls 124 may be positioned over the ends of the main body 104 and coupled with a press-fit or annular snap-fit. The end flange 126 may be ring shaped and extend toward the longitudinal axis and define an opening so that the flange 126 of each end cap 106 108 engages a peripheral edge of an outer end face of each HME filter 110 in order to retain the HME filters 110 between the end caps 106 and 108 and internal retaining features of the main body, for example the tabs 122, as shown in FIGS. 1E and 1G. In some embodiments, for example as shown in FIGS. 5A-5E, discussed in greater detail below. A main body 104 may include a constriction adjacent to a first cylindrical portion 105 on which an HME filter 110 may be seated.

In some embodiments, a monitoring tube 112 extends into the housing assembly 102 and terminates in the central cavity 120. The monitoring tube 112 may extend through an opening defined by the end cap and/or a sidewall of the main body. In some embodiments, for example as shown in FIGS. 1A-1G, the monitoring tube 112 extends into the housing assembly 102 from an end of the main body 104. As shown, one of the end caps 106 may define a sleeve 128 for receiving a portion of the monitoring tube 112. The sleeve 128 may extend away from the main body 104 in a direction approximately parallel (e.g. +/−10 degrees of parallel) to the longitudinal axis 109. The sleeve 128 may be integrally formed with the flange 126 of the end cap 106. In some embodiments, the monitoring tube 112, or a portion thereof, may be integrally formed with the end cap 106 or another portion of the housing assembly 102. In other embodiments, the monitoring tube 112 is coupled within the sleeve with a press-fit so that the monitoring tube 112 does not translate within the sleeve 128 due to forces encountered during normal use. In some embodiments, for example as shown in FIG. 1B, the monitoring tube 112 may extend into the main body 104 between one of the HME filters 110 and an inner wall of the first cylindrical portion 105. The monitoring tube 112 may extend into the central cavity 120 between the two tabs 122 defined by portions of the second cylindrical section, tracheostomy port 114. The tabs 122 may act as guides and limit movement of the monitoring tube 112 within the housing assembly 102.

The HME filters 110 may be generally cylindrical in shape, for example as shown in FIG. 1C. In some embodiments, a monitoring tube 112 may pass through an HME filter 110. In some embodiments, an HME filter 110 may define a channel 202 on a sidewall surface 212 of the cylindrically shaped HME filter 110, as shown for example in FIGS. 2A and 2B. In some embodiments, the HME filter 110 may be made of a compliant material so that a cylindrically shaped sidewall surface 212 complies around a monitoring tube 112 in order to hold the monitoring tube 112 firmly against the main body 104 and form a seal so that respirations in and out of the HME system flow through the HME filters 110. In particular embodiments, the HME filter may be made of one or more of fibers or foamed plastics. The HME filter 110 may include a moisture absorbing substance. The HME filter 110 may prevent small particles, including bacteria and viruses, from entering the tracheostomy tube.

As shown in the perspective view of FIG. 1B, and cross-sectional views of FIGS. 1E and 1G, an open end 130 of the monitoring tube 112 extends into the central cavity 120. In some embodiments, the open end 130 may be proximate to the tracheostomy port 114, as shown in FIG. 1G. In some embodiments, the open end 130 may be coincident with the longitudinal axis 109. As will be discussed in greater detail below, respirations from a patient flowing into and out of the central cavity 120 may be monitored by an external monitoring device via fluidic communication with the central cavity 120 via the open end 130 of the monitoring tube 112.

In some embodiments, the monitoring tube 112, or a portion thereof, may extend through a sidewall of the main body 104 of a housing assembly 102. For example, FIGS. 4A-4F show an embodiment of an HME system 200 similar to the HME system 100 of FIGS. 1A-1G. The HME system 200 may include a housing assembly 102, comprising a main body 104 and two end caps 108. In some embodiments, neither end cap 108 includes a sleeve for a monitoring tube 112, and the monitoring tube 112 may extend through a sleeve 428 formed on the outer sidewall of the main body 104, for example as shown in FIG. 4A. The monitoring tube 112 may extend from the inner sidewall of the main body 104 into the central cavity 120 in a direction perpendicular to the longitudinal axis 109 of the main body 104. The open end 130 of the monitoring tube, as shown in the cross-sectional vies of FIGS. 4D and 4F may coincide with the longitudinal axis 109, and may be positioned between the two HME filters 110.

In some embodiments, the housing assembly 102 may hold the monitoring tube 112 in a curved or bent configuration so that the monitoring tube 112 extends from outside of the housing assembly 102 into the housing assembly 102 in a first direction, then includes a bent or curved portion so that the monitoring tube 112 extends into the central cavity 120 in a second direction, different than the first direction. For example, as shown in FIG. 4D, the monitoring tube 112 enters that sleeve 428 extending in a first direction from outside of the housing assembly 102, and is then curved 90 degrees to then extend into the central cavity 120 in a second direction perpendicular to the first direction.

In some embodiments, it is advantageous for the monitoring tube 112 to extend away from the housing assembly 102 in a direction perpendicular to the axis of the tracheostomy port 114. This configuration prevents the monitoring tube 112 from extending directly away from the patient's neck, e.g. vertically up with the patient lying on their back, which may be uncomfortable for the patient and lead to the monitoring tube 112 pulling on the hub of the tracheostomy tube.

In some embodiments, an HME system may include only one HME filter. In some embodiments, the path of flow through the one or more HME filters may be approximately perpendicular and/or approximately parallel to the axis of the tracheostomy port. For example, as shown in the embodiment of FIGS. 5A-5E, an HME system may include a main body 104 with a first cylindrical portion 105. A single HME filter 110 may be positioned within the first cylindrical portion 105 as shown in the cross-sectional view of FIG. 5D. The main body 104 may include a narrower portion between the first cylindrical portion 105 and the tracheostomy port 114 defining the central cavity 120, as shown in FIG. 5D. The HME filter 110 may be held in place by a removable end cap 108, similarly as discussed above. The path of flow through the single HME filter 110 as shown in FIGS. 5A-5E is parallel to the axis of the tracheostomy port 114.

As shown in FIGS. 5B-5E, the main body 104 may define a sleeve 528 through which the monitoring tube 112 extends into the central cavity 120. The sleeve 528 may be formed integrally with the main body 104 and extend perpendicularly from the axis of the tracheostomy port 114. The monitoring tube 112 may extend through the sleeve 528 and into the central cavity 120 so that the open end 130 of the monitoring tube 112 is positioned proximate to the center of the volume of the central cavity 120 over the tracheostomy port 114, as shown in FIG. 5E.

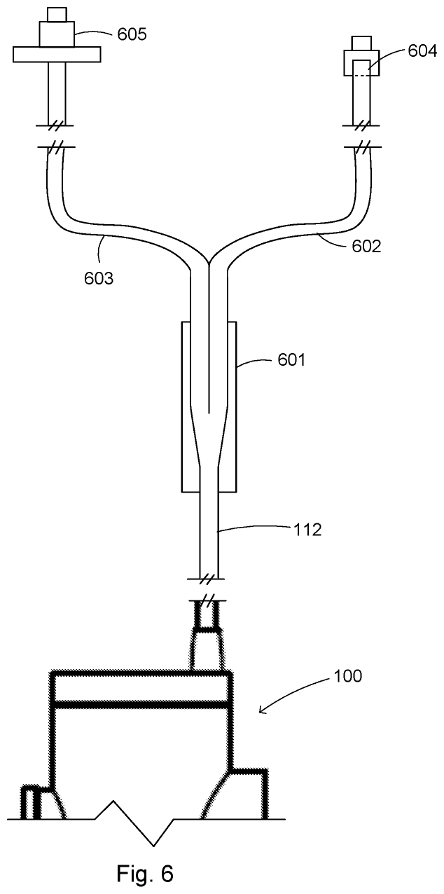

As previously noted, the monitoring tubes 112 of the HME systems as disclosed herein, for example HME systems 100, 200 and 300, may be fluidically connected to one or more respiration monitoring devices. Non-limiting examples of suitable respiration monitoring devices include end tidal carbon dioxide monitors and pressure transducers (e.g., used to measure inhalation and exhalation airflow). The monitoring tube 112 may include one or more connectors opposite the open end 130 for connecting to one or more respiration monitoring devices. In some embodiments, for example as shown in FIG. 6, the monitoring tube 112 may include a bifurcation 601 defining a first split monitoring section 602 and a second split monitoring section 603. The first split monitoring section 602 may include a first connector 604 and the second split monitoring section 603 may include a second connector 605. In particular embodiments, the first connector 604 may be a threaded fitting for connecting to an end tidal carbon dioxide (ETCO2) detector, and the second connector may be a threaded fitting for connecting to a pressure transducer to measure inhalation and exhalation airflow (PTAF).

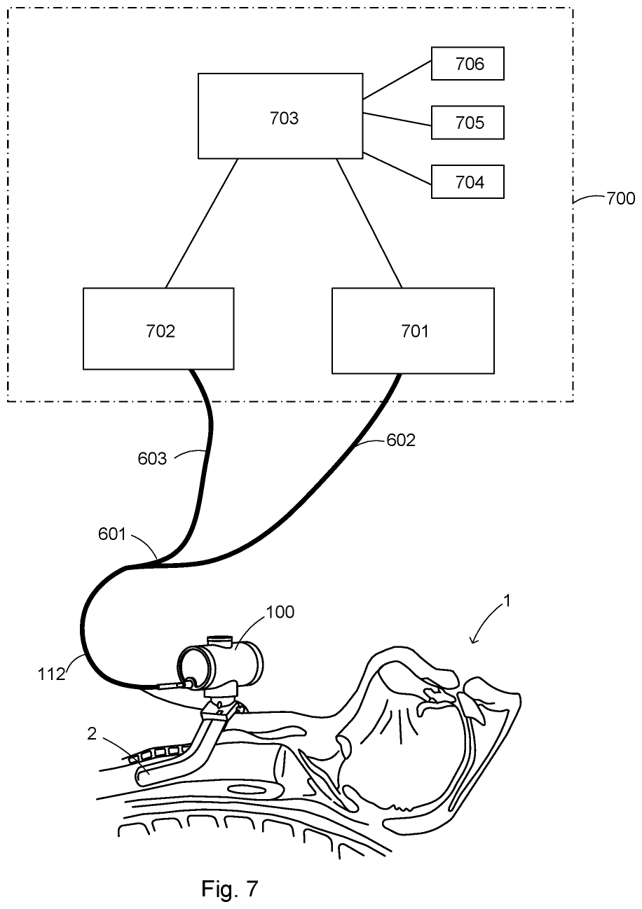

FIG. 7 shows an example sleep study system 700 coupled to an HME system as disclosed herein, for example HME system 100. The sleep study system 700 may include a processor unit 703, for example a PC computer, coupled to one or more monitoring devices (e.g., respiration monitoring devices) for monitoring different parameters of the patient. Non-limiting examples of suitable respiration monitoring devices include end tidal carbon dioxide monitors and pressure transducers (e.g., used to measure inhalation and exhalation airflow). The HME system 100 may be coupled to the hub of a tracheostomy tube 2 inserted into the trachea of a patient 1. The HME system 100 may be used with spontaneously breathing patients. The HME system may be removed if it causes increased work of breathing (e.g., copious, encrusted or dry secretions, or patient's inability to adequately protect their airway).

The bifurcated monitoring tube 112 includes a first split monitoring section 602 connected to an end tidal carbon dioxide (ETCO2) detector 701 and a second split monitoring section 603 connected to a pressure transducer to measure inhalation and exhalation airflow (PTAF) 702. In some embodiments, the monitoring tube may be connected to alternative or additional respiration monitoring devices. During a sleep study, pressure and composition of the patient's respiration are detected at the open end 130 of the monitoring tube 112 in the central cavity 120 of an HME system. Simultaneously during the sleep study, for example polysomnography, the patient may also be connected to other monitoring systems connected to the processor unit 703, including, for example, an electroencephalogram (EEG) monitor 704 to detect brain activity, an electrooculogram (EOG) monitor 705 to detect eye movements, a pulse oximeter or other oximetry device to detect hemoglobin saturation in the patient, and/or an electrocardiogram (ECG) monitor 706 to detect heart activity. These monitoring devices may be used by the processor unit 703 to further evaluate the respiratory and/or other functions of the patient.

During a sleep study, secretions may be emitted by the patient through the tracheal tube into the HME system 100, 200, 300. These secretions may build up and may be periodically removed, for example via suction. During use, the patient may be positioned on their back with the hub of the tracheal tube oriented approximately vertically, and secretions may collect within the central cavity 120 adjacent to the tracheostomy port. During use, the patient may be oriented in other positions, e.g. on their sides, and secretions may collect at one or more low points of the central cavity 120, relative to gravity within the HME cavity. In order to maintain monitoring the respiratory function it is beneficial for the open end 130 of the monitoring tube 112 to not be blocked by secretions. In some embodiments, the open end 130 of the monitoring tube 112 is positioned within the central cavity 120 at a position wherein secretions do not collect. For example, in particular embodiments the open end 130 of the monitoring tube 112 is in a position above, relative to gravity, the lowest points of the central cavity 120 where secretions collect during use. In some embodiments, the open end 130 of the monitoring tube 112 may be positioned proximate to the middle of the tracheostomy port 114, for example as shown in FIGS. 1E, 4C, and 5E. In other embodiments, the open end 130 of the monitoring tube 112 may be positioned at or near the longitudinal axis of the main body. In some embodiments, a portion of the monitoring tube 112 proximate to the open end 130 may be deformable in order to customize the position of the open end 130 based on the patient's sleep position.

In various embodiments, any combination of monitoring tubes 112, including one or more extending through one or more end caps and/or one or more sidewalls of main bodies may be included in an HME system. For example, the main body of FIG. 4A may be used with the end cap with sleeve of FIG. 1A. Similarly, the end cap with sleeve of FIG. 1A could be used with the main body of FIG. 5A, wherein the main body of FIG. 5A may or may not also include a sleeve and monitoring tube. Accordingly, in some embodiments, any of the features HME systems as shown in the figures can be used together in any combination to form embodiments of HME systems as disclosed herein.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims. For instance, any of the examples, alternative examples, etc., and the concepts thereof may be applied to any other examples described and/or within the spirit and scope of the disclosure.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. The phrase “based on” should be understood to be open-ended, and not limiting in any way, and is intended to be interpreted or otherwise read as “based at least in part on,” where appropriate. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate examples of the disclosure and does not pose a limitation on the scope of the disclosure unless otherwise claimed. No language in the specification should be construed as indicating any non-claimed element as essential to the practice of the disclosure.

### Exemplary Embodiments

Exemplary embodiments provided in accordance with the presently disclosed subject matter include, but are not limited to, the claims and the following embodiments.

Some embodiments are related to a heat moisture exchanger (HME) system, for use during a sleep study of a patient with a tracheostomy, the system including a housing assembly defining a central cavity and at least one opening, and comprising a tracheostomy port configured to couple to a hub of a tracheostomy tube of the patient, wherein the tracheostomy port is in direct fluidic communication with the central cavity, at least one HME filter positioned within the housing assembly between the at least one opening and the central cavity, wherein the at least one HME filter is configured to absorb heat and moisture of exhalations from the patient in order to warm and humidify inhalations of the patient flowing from the at least one opening through the HME filter to the tracheostomy port, and a monitoring tube extending from outside of the housing assembly into the housing assembly, wherein the monitoring tube comprises an open end positioned within the central cavity, and wherein the monitoring tube is configured to be connected to a respiration monitoring device in order to detect respiration parameters of the patient during the sleep study.

The at least one HME filter can include a first HME filter and a second HME filter, wherein the at least one opening comprises a first opening and a second opening, the first HME filter is positioned adjacent to the first opening and the second HME filter is positioned adjacent to the second opening, and/or the first HME filter and second HME filter are positioned on opposite sides of the central cavity so that the open end of the monitoring tube is positioned between the first HME filter and the second HME filter. The housing assembly can further include a main body defining the central cavity, and an end cap coupled to the main body adjacent to the first HME filter, wherein the end cap defines a sleeve through which the monitoring tube extends. The monitoring tube can extend from the sleeve to the central cavity between the first HME filter and an inner sidewall of the main body. The first HME filter can be cylindrical and define a channel, and the monitoring tube can be positioned within the channel.

The housing assembly can further include a main body defining the central cavity, and the monitoring tube can extend through a sidewall of the main body into the central cavity. The main body can include a sleeve defining a curved bend, wherein the monitoring tube extends through the sleeve.

At least one HME filter can include a first HME filter, the housing assembly can further include a main body defining the central cavity, the first HME filter can be positioned within the main body, the housing assembly can further include an end cap coupled to the main body adjacent to the first HME filter, and/or the monitoring tube can extends through a sidewall of the main body into the central cavity. The main body can define a first cylindrical portion in which the first HME filter is positioned and a second cylindrical portion, narrower than the first cylindrical portion, defining the central cavity, and the monitoring tube can extend through a sidewall of the second cylindrical portion into the central cavity.

The tracheostomy port can define an axis, and the monitoring tube can extend away from the housing assembly in a direction approximately perpendicular to the axis of the tracheostomy port.

The main body can define a suction port and a cap removably coupled to the suction port configured to allow for removal of secretions from the central cavity.

The monitoring tube can further include at least one connector at an end opposite to the open end within the central cavity, wherein the connector is configured to connect the monitoring tube to the respiration monitoring device. The monitoring tube can further include a bifurcation, wherein the at least one connector comprises a first connector and a second connector, and wherein the first connector is configured to connect to an end tidal carbon dioxide (ETCO2) detector and the second connector is configured to connect to a pressure transducer to measure inhalation and exhalation airflow.

Some embodiments are related to a sleep study system including the HME system and at least one respiration monitoring device fluidically coupled to the monitoring tube and configured to monitor respiration of the patient. At least one respiration monitoring device can include an end tidal carbon dioxide (ETCO2) detector and/or a pressure transducer configured to measure inhalation and exhalation airflow of the patient. It can further include a processor unit, wherein the at least one respiration monitoring device is connected to the processor unit. It can further include at least one of an electroencephalogram (EEG) monitor, an electrooculogram (EOG) monitor, a pulse oximeter, and an electrocardiogram (ECG) monitor connected to the processor unit.

Some embodiments are related to a method of performing a sleep study of a patient with a tracheostomy tube, the method including coupling the tracheostomy port of the HME system of any one of claims 1-13 to a hub of the tracheostomy tube of the patient, coupling the monitoring tube to at least one of an end tidal carbon dioxide detector and a pressure transducer, and detecting end tidal carbon dioxide and/or inhalation and exhalation airflow of the patient as the patient spontaneously breathes with the end tidal carbon dioxide detector and/or the pressure transducer. The monitoring tube can be bifurcated, and the method can further include coupling the monitoring tube to both the end tidal carbon dioxide detector and the pressure transducer.

The method can further include connecting the end tidal carbon dioxide detector and/or the pressure transducer to a processor unit.

The method can further include connecting an electroencephalogram (EEG) monitor, an electrooculogram (EOG) monitor, a pulse oximeter, and/or an electrocardiogram (ECG) monitor to the patient and a processor unit.

1. A heat moisture exchanger (HME) system, for use during a sleep study of a patient with a tracheostomy, the system comprising:
a housing assembly defining a central cavity and at least one opening, and comprising a tracheostomy port configured to couple to a hub of a tracheostomy tube of the patient, wherein the tracheostomy port is in direct fluidic communication with the central cavity;
at least one HME filter positioned within the housing assembly between the at least one opening and the central cavity, wherein the at least one HME filter is configured to absorb heat and moisture of exhalations from the patient in order to warm and humidify inhalations of the patient flowing from the at least one opening through the HME filter to the tracheostomy port; and
a monitoring tube extending from outside of the housing assembly into the housing assembly, wherein the monitoring tube comprises an open end positioned within the central cavity, and wherein the monitoring tube is configured to be connected to a respiration monitoring device in order to detect respiration parameters of the patient during the sleep study.

2. The HME system of claim 1, wherein the at least one HME filter comprises a first HME filter and a second HME filter,
wherein the at least one opening comprises a first opening and a second opening,
wherein the first HME filter is positioned adjacent to the first opening and the second HME filter is positioned adjacent to the second opening, and
wherein the first HME filter and second HME filter are positioned on opposite sides of the central cavity so that the open end of the monitoring tube is positioned between the first HME filter and the second HME filter.

3. The HME system of claim 2, wherein the housing assembly further comprises a main body defining the central cavity, and an end cap coupled to the main body adjacent to the first HME filter, and
wherein the end cap defines a sleeve through which the monitoring tube extends.

4. The HME system of claim 3, wherein the monitoring tube extends from the sleeve to the central cavity between the first HME filter and an inner sidewall of the main body.

5. The HME system of claim 4, wherein the first HME filter is cylindrical and defines a channel, and
wherein the monitoring tube is positioned within the channel.

6. The HME system of claim 1, wherein the housing assembly further comprises a main body defining the central cavity, and
wherein the monitoring tube extends through a sidewall of the main body into the central cavity.

7. The HME system of claim 6, wherein the main body comprises a sleeve defining a curved bend, and
wherein the monitoring tube extends through the sleeve.

8. The HME system of claim 1, wherein the at least one HME filter comprises a first HME filter,
wherein the housing assembly further comprises a main body defining the central cavity,
wherein the first HME filter is positioned within the main body,
wherein the housing assembly further comprises an end cap coupled to the main body adjacent to the first HME filter, and
wherein the monitoring tube extends through a sidewall of the main body into the central cavity.

9. The HME system of claim 8, wherein the main body defines a first cylindrical portion in which the first HME filter is positioned and a second cylindrical portion, narrower than the first cylindrical portion, defining the central cavity, and
wherein the monitoring tube extends through a sidewall of the second cylindrical portion into the central cavity.

10. The HME system of claim 1, wherein the tracheostomy port defines an axis, and wherein the monitoring tube extends away from the housing assembly in a direction approximately perpendicular to the axis of the tracheostomy port.

11. The HME system of claim 3, wherein the main body defines a suction port and a cap removably coupled to the suction port configured to allow for removal of secretions from the central cavity.

12. The HME system of claim 1, wherein the monitoring tube further comprises at least one connector at an end opposite to the open end within the central cavity, and
wherein the connector is configured to connect the monitoring tube to the respiration monitoring device.

13. The HME system of claim 12, wherein the monitoring tube further comprises a bifurcation,
wherein the at least one connector comprises a first connector and a second connector, and
wherein the first connector is configured to connect to an end tidal carbon dioxide (ETCO2) detector and the second connector is configured to connect to a pressure transducer to measure inhalation and exhalation airflow.

14. A sleep study system comprising:
a heat moisture exchange (HME) system comprising:
a housing assembly defining a central cavity and at least one opening, and comprising a tracheostomy port configured to couple to a hub of a tracheostomy tube of the patient, wherein the tracheostomy port is in direct fluidic communication with the central cavity;
at least one HME filter positioned within the housing assembly between the at least one opening and the central cavity, wherein the at least one HME filter is configured to absorb heat and moisture of exhalations from the patient in order to warm and humidify inhalations of the patient flowing from the at least one opening through the HME filter to the tracheostomy port; and
a monitoring tube extending from outside of the housing assembly into the housing assembly, wherein the monitoring tube comprises an open end positioned within the central cavity, and wherein the monitoring tube is configured to be connected to a respiration monitoring device in order to detect respiration parameters of the patient during the sleep study; and
at least one respiration monitoring device fluidically coupled to the monitoring tube and configured to monitor respiration of the patient.

15. The sleep study system of claim 14, wherein the at least one respiration monitoring device comprises an end tidal carbon dioxide (ETCO2) detector and/or a pressure transducer configured to measure inhalation and exhalation airflow of the patient.

16. The sleep study system of claim 14, further comprising a processor unit, wherein the at least one respiration monitoring device is connected to the processor unit.

17. The sleep study system of claim 16, further comprising at least one of an electroencephalogram (EEG) monitor, an electrooculogram (EOG) monitor, a pulse oximeter, and an electrocardiogram (ECG) monitor connected to the processor unit.

18. A method of performing a sleep study of a patient with a tracheostomy tube, the method comprising:
providing a heat moisture exchanger (HME) system, for use during a sleep study of a patient with a tracheostomy, the HME system comprising:
a housing assembly defining a central cavity and at least one opening, and comprising a tracheostomy port configured to couple to a hub of a tracheostomy tube of the patient, wherein the tracheostomy port is in direct fluidic communication with the central cavity;
at least one HME filter positioned within the housing assembly between the at least one opening and the central cavity, wherein the at least one HME filter is configured to absorb heat and moisture of exhalations from the patient in order to warm and humidify inhalations of the patient flowing from the at least one opening through the HME filter to the tracheostomy port; and
a monitoring tube extending from outside of the housing assembly into the housing assembly, wherein the monitoring tube comprises an open end positioned within the central cavity, and wherein the monitoring tube is configured to be connected to a respiration monitoring device in order to detect respiration parameters of the patient during the sleep study;
coupling the tracheostomy port of the HME system to the hub of the tracheostomy tube of the patient;
coupling the monitoring tube to at least one of an end tidal carbon dioxide detector and a pressure transducer; and
detecting end tidal carbon dioxide and/or inhalation and exhalation airflow of the patient as the patient spontaneously breathes with the end tidal carbon dioxide detector and/or the pressure transducer.

19. The method of claim 18, wherein the monitoring tube is bifurcated, and wherein the method further comprises coupling the monitoring tube to both the end tidal carbon dioxide detector and the pressure transducer.

20. The method of claim 18, further comprising:
connecting the end tidal carbon dioxide detector and/or the pressure transducer to a processor unit; and/or
connecting an electroencephalogram (EEG) monitor, an electrooculogram (EOG) monitor, a pulse oximeter, and/or an electrocardiogram (ECG) monitor to the patient and a processor unit.

21. (canceled)
